(19) United States
(12) Patent Application Publication
(10) Pub. No.: US 2026/0198810 A1
(43) Pub. Date: Jul. 16, 2026

(54) A BLOOD LOSS DETECTION METHOD AND SYSTEM

(71) Applicant: ISTANBUL MEDIPOL UNIVERSITESI TEKNOLOJI TRANSFER OFISI ANONIM SIRKETI, Beykoz, Istanbul (TR)

(72) Inventors: Yunus Oktay ATALAY, Beykoz, Istanbul (TR); Semih MACIT, Beykoz, Istanbul (TR); Osman MERSIN, Beykoz, Istanbul (TR); Kevser Banu KOSE, Beykoz, Istanbul (TR)

(21) Appl. No.: 19/134,818

(22) PCT Filed: Nov. 8, 2023
(86) PCT No.: PCT/TR2023/051283

§ 371 Date: Jun. 2, 2025

(30) Foreign Application Priority Data
Dec. 2, 2022 (TR) 2022/018456

(51) Int. Cl. A61B 5/1455; A61B 5/00; A61B 5/145

(52) U.S. Cl. CPC A61B 5/1455; A61B 5/14546; A61B 5/7225; A61B 5/7246; A61B 2560/0462

(57) ABSTRACT

The invention relates to a method and a system for said method for quantifying the amount of blood loss in the environment in which the bleeding occurs and/or on the patient who has bleeding.

### TECHNICAL FIELD

The invention relates to a system and a method for determining the amount of blood loss in the environment where the bleeding occurs and/or on the patient who has bleeding.

### STATE OF THE ART

Blood loss is a decrease in the volume of blood in the housing of the living thing caused by blood flowing out of open wounds in the housing. This situation mostly occurs during surgeries or accidents. Blood loss in accidents is an accident and should be treated urgently, but blood loss during surgery is very normal. Because this blood loss is mostly under the control of the doctor and the lost blood is also returned to the housing.

However, surgeries such as organ transplantation, open heart surgery, orthopedic prosthesis surgeries, brain surgery, and tumor surgeries are surgeries with a high probability of bleeding. If a patient has excessive blood loss during surgery, the risks of mortality and morbidity increase dramatically. Experts think that increasing the accuracy and reliability of blood loss prediction will reduce the results of surgeries in which such bleeding occurs.

These hemorrhages have negative effects on the patient's hemodynamics such as hypertension, impaired organ perfusion, and even death, and the lack of a valid detection mechanism for intraoperative blood loss causes an increase in the rate of sepsis (severe infection), length of stay in the intensive care unit, and especially mortality. According to the research, approximately 40% of trauma deaths are caused by bleeding. In addition, another study shows that improper intraoperative transfusion may result in an increased risk of mortality and morbidity. Therefore, rapid control of bleeding and correct blood transfusion is very important for the health of the patient during and after the operation.

Therefore, one of the most important tasks of anesthesiologists during surgery is to try to keep the patient's hemodynamics (vital signs, heart rate, arterial blood pressure, blood oxygen level) stable throughout the surgery. In addition to causing a volume deficit in the patient, blood loss can also adversely affect oxygen delivery to especially important organs. In order to replace the volume deficit, fluid therapy is applied to the patient, and when the hemoglobin level falls below a certain threshold value, it is necessary to give blood products. However, it is of great importance to accurately measure and evaluate the amount of bleeding during surgery, both when performing fluid replacement and when performing blood transfusion. Because unnecessary fluid will cause loading, while a small amount of fluid will cause further deterioration of hemodynamics.

However, today, where technological development is so far ahead, bleeding calculation methods, which have been used for years, are still used. Unfortunately, these methods only give an estimated value, in fact, treatments against bleeding of patients are also made through these calculations; this calculation may also vary between individuals. Therefore, there is a need for methods that measure the amount of bleeding more precisely.

The most common method of estimating blood loss is visual estimation. However, according to the applications, methods such as gravimetric method, colorimetric method, calculation method, and direct method are used.

Visual estimation is the most studied method as well as the most commonly used method by clinicians. With this method, it is tried to calculate how much blood is in the blood-sucking towel-like material such as a blood-soaked sponge and compress, how much blood is in the mixture of blood and liquid in the aspirator (by asking the nurse how much fluid she gives, which nurses often tell an estimate of the amount of fluid given), the surgical cover and the amount of blood in the stretcher. As for how much blood the sponge and the abdomen can absorb, it is assumed that a whole blood-soaked sponge contains 10 mL of blood, and the compresses will contain about 100 mL of blood in the whole blood-soaked state, as a general opinion determined by trial. According to the result of the estimation, the patient is given a blood transfusion. Even if pictograms are used to develop this method, factors such as professional experience, sex, and age do not provide a clear improvement in the accuracy of this prediction. Calculating the amount of bleeding occurs when the person who visually evaluates the amount of blood in these materials evaluates it as a possible amount (for example, 30% of the sponge seems to be bloody), which naturally causes inconsistencies in the calculation of bleeding and, in fact, the real value cannot be known.

Another method is the gravimetric method, which indirectly measures blood loss. This method is a weight-based measurement method. It is obtained by subtracting the dry weight of the material from the weight obtained by weighing the surgical material contaminated with blood during the surgery. In addition, the volume of the solution (blood rinse) collected in the aspirator with the blood is estimated and the calculation is made according to the assumption that 1 mL of blood is equal to 1 gram. Although this method provides a more quantitative estimation result causes a great error in the calculation, especially since the liquid mixed in the blood causes the blood to be diluted.

The method based on the colorimetric measurement is Triton, a smartphone application developed by Gauss Surgical, the amount of Hemoglobin (Hb) used with this application is determined by taking photographs of surgical sponges, gauze and cans used thanks to the machine learning algorithm. In this method, blood loss can be estimated by entering preoperative blood data. According to the research, it is considered to be the most accurate method for blood loss estimation since it gives the most accurate results with the blood volumes used as a reference. However, it could not go beyond giving estimated and average results to the authorities, even though it was expressed in this way.

Calculation methods are estimation methods based on formulas in which estimated blood loss is determined. The mathematical approach based on formulas is based on anthropometry and hematology parameters. The basic principle of this method is to estimate blood loss by multiplying the estimated blood volume of the patient by the inequality in the hemoglobin ratio. However, since the plasma volume may change unexpectedly over time and blood loss changes the diluting effect, formulas containing hematological parameters do not give reliable results. Although it is tried to predict blood loss with formulas based on hemoglobin mass loss, which is claimed to be more reliable than the blood volume loss theory, each estimation formula needs much more research and clinical trials for perioperative reliability. The most frequently used formula in the literature is the Nadler formula. This formula includes parameters such as gender, height, and weight in the calculation. However, since they calculate the total amount of blood higher than the actual amount, all of these calculations need to be improved.

The other method used in blood loss prediction is known as direct spectrophotometry. Briefly, in this method, the blood sample during surgery is extracted from surgical equipment (surgical sponges and surgical drums), and the amount of direct Hb in the samples during surgery is analyzed in terms of postoperative light absorption spectrometry after the sample is dissolved in 5% sodium hydroxide solution. However, this method is time-consuming for emergency surgeries and much costlier to predict blood loss. This statement and the 10% error rate detected in this method indicate why the direct spectrophotometry method is rarely preferred in predicting blood loss.

Although there are many different methods for predicting blood loss in the literature, there is no quantitatively precise routine. This complicates the situation of clinicians. Therefore, a new technology or approach should be developed to determine the exact perioperative blood loss with great accuracy in real time. Providing the right amount of blood supplement can be important both to avoid unnecessary blood supplementation and to reduce the patient's excess blood product intake. It is strongly recommended to use the necessary amount of blood products all over the world and to avoid unnecessary use of blood products.

All the problems mentioned above have made it necessary to make an innovation in the relevant field as a result.

### Objectives and Brief Description of the Invention

The main objective of the invention is to ensure that the amount of blood loss in the patient is determined quantitatively.

The present invention relates to a computer-applied hemoglobin concentration determination method to determine the amount of blood loss in the patient to meet the abovementioned needs and objectives. Accordingly, the present method comprises the steps of providing electrical excitation signals for thermal radiation values after the sample has been excited by the light source, read by a radiation measurement sensor, correlating the signal obtained to calculate the hemoglobin concentration based on predetermined electrical signal to hemoglobin concentration ratios.

Thus, the concentration of suspended and homogenized hemoglobin in the blood can be determined and the volume of the sample can be easily found over this value.

In a preferred embodiment of the invention, an electrical intersection signal is provided for the ambient thermal radiation values, and the electrical concentration signal is found by subtracting the intersection signal and the excitation signals from each other, and the electrical concentration signal is correlated based on the electrical signal-hemoglobin concentration ratios.

Since all substances will currently emit radiation and the radiation measurement sensor can be collected by the sensor in these radiations, first of all, the ambient values are measured, and these are decomposed before the final detection. Thus, more precise results are obtained that are free from ambient effects.

In a preferred embodiment of the invention, the correlation step is performed by a neural network algorithm trained with predetermined electrical signal-hemoglobin concentration data. In a preferred embodiment of the invention, the light source excitation wavelength is 805 nm. Thus, it was made possible to provide equal absorption of both oxyhemoglobin and deoxyhemoglobin.

### DEFINITIONS OF FIGURES

The figures and related descriptions used to better explain the device developed by this invention are as follows.

FIG. 1. A representative schematic view of an embodiment of the system of the invention. FIG. 1 a. A representative image showing the sample stimulation and response steps.

FIG. 1 b. A flow diagram of the method of the invention.

FIG. 2. A top view of an embodiment of the system of the invention.

FIG. 2 a. A sub-image of an embodiment of the system of the invention.

### DEFINITIONS OF COMPONENTS/PIECES/PARTS OF THE INVENTION

In order to better explain the device developed by this invention, the parts and pieces in the figures are numbered, and corresponding numbers are given below.
10. Light source
20. Radiation measurement sensor
21. Filter
30. Distance sensor
40. Amplifier
50. Conditioner
60. Display
70. Button
P. Processing unit
S. Sample
G. Housing
K. Panel
R. Rotational axis

### Detailed Description of the Invention

The subject matter of the invention relates to a system for quantifying the amount of blood loss in the environment in which the bleeding occurs and/or on the patient who has bleeding and a method for said system.

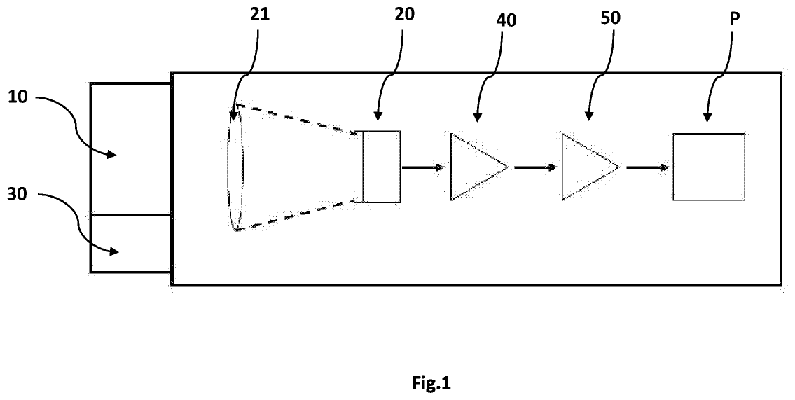

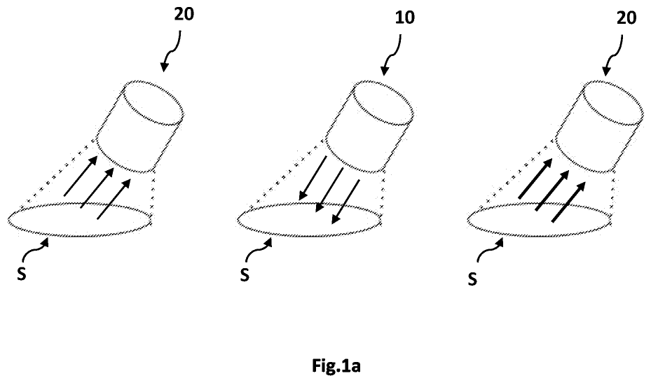

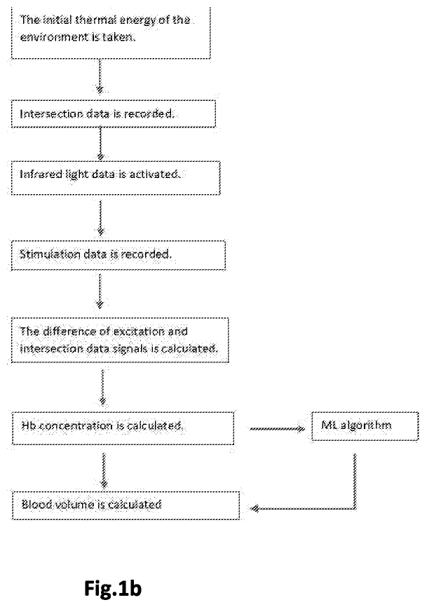

Referring to FIG. 1-1b, the basis of the hemoglobin detection method and system of the invention is the stimulation of a blood sample (N) under a light and the measurement of the thermal radiation emitted by the hemoglobin of the blood sample (S) against this stimulation and the determination of the hemoglobin concentration after converting the measured value into an electrical value and correlating it accordingly.

Accordingly, at least one light source (10) and at least one radiation measurement sensor (20) are used for the relevant detection process. When light is applied with the light source (10) of the blood sample (S), the hemoglobin of the blood sample(S) absorbs this ray. Here, the absorbed waves are then emitted back as thermal radiation by the hemoglobin. Here, the radiation measurement sensor (20) captures the thermal radiation and converts it into electrical values (voltage, current, etc.). These electrical values are called excitation signals. Preferably, the excitation signals are kept in a two-dimensional list containing time-electrical value values. The infrared temperature sensor or thermobattery sensor may be selected as the radiation measurement sensor (20) in a preferred embodiment.

In a preferred embodiment, the wavelength of the light source is selected as a value in which oxyhemoglobin and deoxyhemoglobin provide equal absorption so that both oxyhemoglobin and deoxyhemoglobin can be measured in a single phase and the oscillations of different substances do not interfere with the measurement. In the experiments, it was observed that this equal absorption condition was met at 260, 340, 390, 423, 453, 500, 530, 545, 570, 585, and 805 nm wavelengths. Preferably, the light source is of the infrared type.

The values obtained here are correlated by a processing unit (P) based on the predetermined electrical value-hemoglobin concentration ratios and there is a hemoglobin concentration.

Here, the correlation process can be provided directly by proportioning over the predetermined electrical value-hemoglobin concentration value, but preferably by artificial neural networks, preferably carried out by the processing unit (P). Here, the ratio between the hemoglobin concentration obtained in the preclinical trials and the time-voltage ratio and the hemoglobin concentration will be determined. In this section, unknown parameters (weights, W) and unobservable random errors () required for the machine learning algorithm are detected by the ML algorithm. The digital data stored in the hemoglobin concentration signal are equipped with artificial neural network algorithms and linearized. The quantitatively predicted hemoglobin concentration is then obtained, and the data are stored for use in the calculation of blood volume. Here, preferably, the artificial neural network is trained as follows; a data set is created by examining the blood sample with a known hematocrit (HCT) value and certain volumes with the relevant system. This data set will include Hb concentration, blood volume, voltage, or current (sensor-dependent) parameters. The main relationship will be the connection between Hb concentration and voltage or current. Although it is not restrictive, the polynomial regression algorithm is used for this procedure. Here, it is known that different algorithms give satisfactory results among such correlation data.

In addition, the data obtained in the detection process can be given to the machine learning process as a data set and it can be ensured that the artificial neural network gives more accurate results.

The measurement results obtained are preferably recorded in a non-volatile memory unit. It can be stored in the memory unit mentioned in other information such as measurement time, date, and the results.

In a preferred embodiment, the thermal radiation of the medium is detected before the light is applied to the blood samples(S) as shown in Figure la. In this method, the radiation measurement sensor (20) is activated before the light source (10). The thermal radio of the medium is measured for a while and then the light source (10) is activated. Accordingly, ambient thermal radiation values including electrical signals, also called “intersection signals”, are obtained from the first thermal radiation detection read from the radiation measurement sensor. Preferably, the intersection signal is kept in a two-dimensional list containing time-electrical value values.

Following the detection of the intercepted signal, excitation is provided on the sample(S) via the light source (10) as described above and excitation signals are obtained. Then, by subtracting the intercept signals from said excitation signals, the environmentally free signals are obtained by proportioning them to obtain the hemoglobin concentration as described above or by means of artificial neural networks. Preferably, the rate of change can then be proportioned according to the experimental findings of the preclinical data.

Preferably, after the hemoglobin concentration value is obtained, this value is proportioned to obtain the volume of blood. Red blood cells that contain hemoglobin are found suspended and homogeneously distributed in the blood plasma. This information indicates that hemoglobin is found in equal concentrations in 1 mL of blood volume. Therefore, in light of the preclinical trials and the hemoglobin concentration obtained, the total volume of the measured sample will be calculated, proportional to the hemoglobin concentration per 1mL of blood volume. At this stage, the hemoglobin concentration per 1 mL blood volume will be optionally optimized with the HCT value to be routinely taken from the patient before the surgery and the accuracy rate of the procedure will be increased specifically for each patient. For patients without HCT (for example, in measurements to be performed for patients without surgical conditions), the general, pre-clinical hemoglobin concentration, which is determined and accepted to be unchanged, will be automatically used.

Here, the processing of the data received from the radiation measurement sensor (20) and preferably the operation of the light source (10) radiation measurement sensors (20) is carried out by a processing unit (P). The processing unit (P) is configured to perform said operations. The processing unit (P) needs appropriate instructions to perform according to the method steps described in the system. The aforementioned instructions are customized for said system and method. Said instructions may be contained in a program or a machine-readable medium containing said program. In addition, said instructions may be stored in a memory unit in the system or provided as embedded, where said processing unit (P) and the memory unit may be integrated.

In the said system, a selective filter (21) is used so that the radiation measurement sensor (20) can only, at least mostly, detect the thermal radiation emitted from hemoglobin and eliminate the thermal radiation of other objects. The selective filter (21) is preferably integrated with the radiation measurement sensor (20). Said filter (21) is preferably configured to allow radiation transition at a wavelength of 805 nm.

Moreover, the system may also include a distance sensor (30). In order to ensure accurate measurement, the distance between the radiation measurement sensor (20) and the sample is important. Here, a distance sensor (30) checks whether this distance is provided correctly. Preferably, the data received from the distance sensor (30) can be used as the condition for starting the light source (10) and/or the radiation measurement sensor (20). In such a conditioning, measurement or excitation is not performed if the desired distance range is not provided.

An electrical amplifier (40) may be provided between the radiation measurement sensor (20) and the processing unit (P). The amplitude of the measured electrical signals may be low, resulting in data loss and inaccurate analysis of electrical properties. Therefore, the raw signal derived directly from the sample is amplified using operational amplifiers. Amplification will increase computational efficiency, and accuracy, and also enable the development of more efficient algorithms for machine learning applications.

Preferably, the system further includes an electrical conditioner (50). It is in three main parts:
signal conditioning, amplification, isolation, and filtering. The main purpose of using signal conditioning is to increase transaction reliability. With signal conditioning, the amplified electrical signal will be standardized and improve troubleshooting. Also, mismatched signal problems will be resolved.

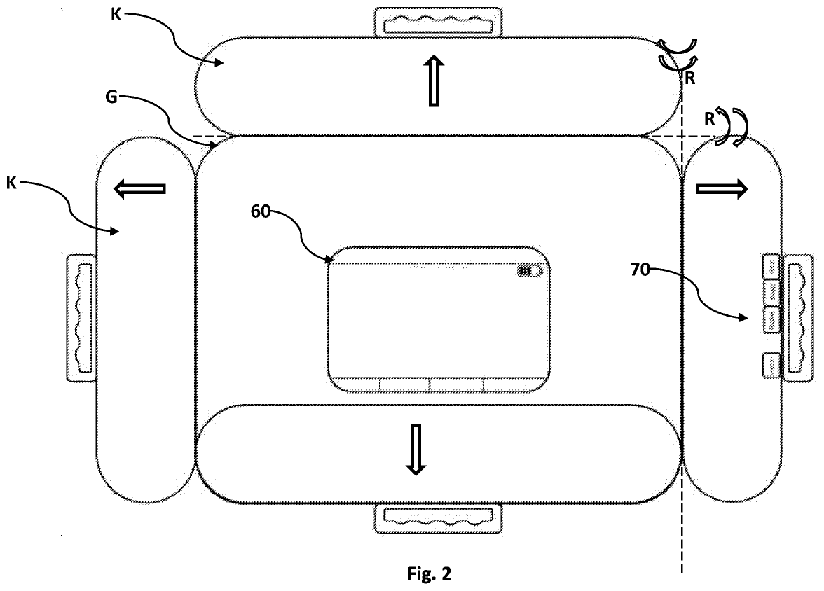

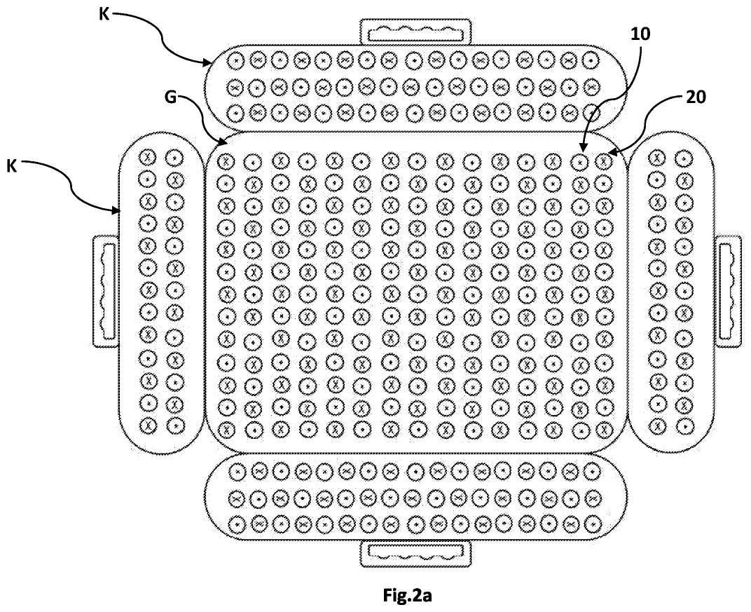

Referring to FIGS. 2 and 2a, the said system can also be presented as an integrated device. The device is configured on a housing (G). Preferably, a display (60) is located on the housing (G) to show the hemoglobin concentration or volume information. Alternatively, instead of using a display (E), the hemoglobin concentration or volume information may be transmitted wired or wirelessly to another device.

There is at least one, preferably multiple, light source (10) and radiation measurement sensor (20) on one side of said housing (G). The light source (10) and the radiation measurement sensor (20) are arranged sequentially, that is, there is a radiation measurement sensor (20) continuously next to a light source (10). In this way, it is possible to provide both stimulation and reading homogeneously.

In a preferred embodiment of the invention, there are panels (K) with at least one preferably multiple on the edges of the housing (G), especially at least one on each edge. Here, the panels (K) are pivotally connected to the housing (G). It may be provided with respect to an axis of rotation (R) extending along the pivot edge. Here, it is possible to connect the panels (K) to the housing (G) with joint-like elements. Normally, the sample surface to be measured may not always have a standard area. Therefore, foldable panels (K) provide ease of measurement suitable for any area. In case of deficiency, errors and missing data will be encountered in the measurements taken for the measurement surfaces that have more area than the device measurement area.

Said panels (K) are provided as sliding and movable in the direction of the linear arrows in FIG. 2. Here, sliding panels (K) can be provided directly on the housing (G), as well as recessed panels (K) with the feature of being able to enter the housing (G) can be provided through the slots provided in the said housing (G).

The housing (G) may also have various buttons (70) to use the various functions of the device. These buttons (70) may be configured to start and finish the on, off, and measurement process. In addition, some of the buttons (70) may be customized for multiple or single measurement processes.

In multiple measurements, multiple measurements can be taken in a row and the aggregate results of these measurements are reported to the user. This is done in specific cycles and the user is informed about these cycles. For example, after this option is selected, the system repeats itself continuously until the user presses the finish button (70) (If it presses Start again after the first initial press, it puts itself on hold. For example, a user who wants to perform multiple measurements can press the start button (70) again after completing the first measurement and put the device in standby mode. When the blood sample to be measured arrives, you can perform the procedure by pressing the start button again. If they repeat this procedure, they can take countless measurements.) and perform countless measurements on request. In a single measurement, a measurement is taken after pressing the start button (70). When the procedure is completed, it terminates all procedures, records the blood volume, time, date, and measurement time in its permanent memory, and notifies the user of the single measurement result. When the multiple measurement option is activated, this option is automatically switched to off mode.

Another button (70) may also be assigned for the calibration process. In this function, the device continuously tests itself with the recorded data and parameters embedded in the system, and if notification of calibration is received by the user and the device, the system calibrates with the entered parameters. Here, hematocrit value updates of patients with special needs can also be made with this option.

The present application further comprises a device comprising elements for performing the method of claims 1-10 when executed by a processing unit, a program comprising instructions for performing the method of claims 1-10 when executed by a processing unit, and a computer-readable medium comprising said program.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A computer-implemented oxyhemoglobin and deoxyhemoglobin concentration detection method to determine the amount of blood loss in the patient, comprising the following steps:
measuring the distance between a radiation measurement sensor and a sample;
starting a light source and/or the radiation measurement sensor based on the data received from a distance sensor;
providing electrical excitation signals read by the radiation measurement sensor for thermal radiation values of the sample after excitation by the light source wherein the light source wavelength is 805 nm;
correlating the obtained signal by artificial neural networks to calculate the hemoglobin concentration based on the predetermined electrical signal-hemoglobin concentration ratios.

29. A method according to claim 28, wherein the wavelength of said light source is selected as a wavelength in which both oxyhemoglobin and deoxyhemoglobin show equal absorption.

30. A method according to claim 28, wherein said light source is of infrared type.

31. A method according to claim 28, wherein an electrical intercept signal is provided for ambient thermal radiation values read by a radiation measurement sensor before the sample is excited by the light source, and the intercept signal and the excitation signals are subtracted from each other to provide an electrical concentration signal, and the electrical concentration signal is correlated based on electrical signal to hemoglobin concentration ratios.

32. A method according to claim 31, wherein the neural network is trained with predetermined electrical signal-hemoglobin concentration data.

33. A method according to claim 28, wherein the correlation step is carried out by proportioning the concentration signal obtained with the predetermined electrical signal-hemoglobin concentration ratios.

34. A method according to claim 28, wherein the volume of the measured sample is calculated by correlating with the obtained hemoglobin concentration.

35. A method according to claim 34, wherein the hemoglobin concentration is correlated with the hematocrit value for volume determination.

36. A deoxyhemoglobin and oxyhemoglobin concentration detection system for determining the amount of blood loss in a patient, comprising the following:
at least one light source to provide excitation to a sample;
at least one radiation measurement sensor to measure the waves emitted from said sample;
at least one distance sensor to check the distance between the radiation measurement sensor and the sample;
at least one processing unit configured to operate a method according to claim 28.

37. A system according to claim 36, wherein said light source is configured to provide a beam at a wavelength in which both oxyhemoglobin and deoxyhemoglobin are equally absorbed.

38. A system according to claim 36, wherein said light source is of infrared type.

39. A system according to claim 36, comprising a housing on which said light source and said radiation measurement sensor are positioned.

40. A system according to claim 36, wherein said light source and the radiation measurement sensor are positioned on the same surface.

41. A system according to claim 40, wherein said light source and the radiation measurement sensor are positioned sequentially.

42. A system according to claim 39, comprising panels provided at the edges of said housing and rotatably provided along the connection axis.

43. A system according to claim 39, comprising a light source and a radiation measurement sensor on said panels.

44. A system according to claim 36, wherein the radiation measurement sensor is a thermobattery sensor.

45. A system according to claim 36, wherein the radiation measurement sensor is an infrared temperature sensor.

46. A system according to claim 36, wherein said light source is configured to operate at a wavelength of 805 nm.

47. A system according to claim 36, comprising a selective filter to prevent said radiation measurement sensor from receiving radiations other than radiations.

48. A system according to claim 36, wherein the distance sensor is provided for measuring a distance between said infrared temperature sensor and/or said radiation measurement sensor and said measured area.

49. A system according to claim 48, wherein said distance sensor is of ultrasonic type.

50. A system according to claim 36, comprising an amplifier for amplifying the radiation measurement sensor data converted into said electrical signal.

51. A system according to claim 36, comprising a signal conditioner.

52. A computer program for detecting deoxyhemoglobin and oxyhemoglobin concentration to determine the amount of blood loss on the patient, comprising instructions that, when executed by the processing unit, enable the system and method according to claims 36.

53. A computer-readable medium for detecting deoxyhemoglobin and oxyhemoglobin concentration to determine the amount of blood loss on the patient, comprising instructions that, when executed by the processing unit, enable the system and method according to claim 36.
